(19) United States
(12) Patent Application Publication
(10) Pub. No.: US 2016/0030247 A1
(43) Pub. Date: Feb. 4, 2016

(54) MULTI-USE, FLEXIBLE MEDICAL BANDAGE

(71) Applicant: Beekley Corporation, Bristol, CT (US)

(72) Inventor: Ryan Joseph Salvino, Burr Ridge, IL (US)

(21) Appl. No.: 14/790,858

(22) Filed: Jul. 2, 2015

Related U.S. Application Data
(60) Provisional application No. 62/030,723, filed on Jul. 30, 2014.

(51) Int. Cl. A61F 13/00; A61F 13/02; A61L 15/52; A61L 15/44; A61L 15/58

(52) U.S. Cl. CPC A61F 13/00063; A61L 15/44; A61L 15/58; A61L 15/52; A61F 13/023; A61F 2013/00285

(57) ABSTRACT

A multi-use bandage has a flexible fabric and a pouch forming a medication insert area located on an underside of the flexible fabric. The medication insert area is configured for overlying a treatment area on a user and for receiving therein any of a plurality of different medications for treating any of a plurality of different symptoms. An adhesive is located on an underside of the flexible fabric and surrounds the medication insert area. The adhesive is configured for adhesively engaging the user's skin about the treatment area, and for preventing medication inserted in the medication insert area from spreading or moving from the treatment area to an unselected area of treatment.

### CROSS-REFERENCE TO RELATED APPLICATIONS

This patent application claims benefit under 35 U.S.C. §119 to U.S. Provisional Patent Application No. 62/030,723, filed Jul. 30, 2014, which is hereby incorporated herein by reference in its entirety as part of the present disclosure.

### FIELD OF THE INVENTION

The present invention is directed to medical bandages, and more particularly, to multi-use flexible medical bandages that can receive any of numerous different medications for treating any of numerous different symptoms or conditions.

### BACKGROUND INFORMATION

Medical bandages for covering cuts, abrasions, wounds or other conditions on the surface of the skin are well known. A typical medical bandage consists of a tape made of a thin plastic or cloth material with an adhesive on one side to releasably attach the medical bandage to the surface of the skin. A pad made of gauze or other material is fixed to the medical bandage on the side having the adhesive, typically in the approximate center area of the bandage. A releasable backing is provided over the adhesive. In use, the pad is typically placed over the cut or wound, the releasable backing is removed, and the adhesive tape is fixed to the skin to hold the pad over the cut or wound while it heals.

In some cases, a patient or physician may want to apply a medication on the skin and underneath a bandage. For example, in the case of a cut or abrasion, an antibiotic cream or gel may be applied to the skin. Alternatively, a patient or physician may want to hold a medication in place on the skin, such as when treating a fungal infection of the skin with an ointment, cream, gel, foam or lotion. In these cases, the patient or physician must place the ointment, cream, gel foam or lotion directly on the skin or on the pad on the bandage before the bandage is fixed to the skin. As pressure is applied to fix the bandage to the skin, the medication can flow outward from underneath the bandage. This can be messy and result in the medication being spread over portions of the skin that are not being treated. In addition, the bandage may not adequately hold the medication in place if the patient engages in physical activity after application of the bandage.

Medical bandages having a recessed area in the pad holding a particular medication have been developed to address some of these concerns. For example, bandages with a circular recess containing salicylic acid to treat warts are known. However, these bandages cannot be used to apply or provide medications selected by the patient or physician, as the medication is provided with the bandage. In addition, these bandages do not adequately contain the medication and it can spread to unintended areas on the skin either during application of the bandage or thereafter, particularly as the user engages in physical activity.

It is an object of the present invention to overcome one or more of the above-described drawbacks and/or disadvantages of the prior art.

### SUMMARY OF THE INVENTION

In accordance with a first aspect, the present invention is directed to a multi-use bandage comprising a flexible fabric, and a medication insert area located on an underside of the flexible fabric and defining a plurality of sides. The medication insert area is configured for overlying a treatment area on a user and for receiving therein any of a plurality of different medications for treating any of a plurality of different symptoms. An adhesive is located on an underside of the flexible fabric and surrounds a plurality of sides of the medication insert area. The adhesive is configured for adhesively engaging the user's skin about the treatment area, and for preventing medication inserted in the medication insert area from spreading or moving from the treatment area to an unselected area of treatment.

In some embodiments of the present invention, a pouch defines the medication insert area. In some embodiments, the pouch includes a waterproof surface located on the underside of the flexible fabric, and a mesh overlying the waterproof surface and configured to receive any of the plurality of different medications between the mesh and waterproof surface. In some such embodiments, the multi-use bandage further comprises a waterproof membrane defining the waterproof surface. In some embodiments, the mesh forms a flap. In some embodiments, the pouch includes a mouth. The mouth is preferably configured for inserting therethrough a medication into the medication insert area. In some such embodiments, the multi-use bandage includes an adhesive on the mouth of the pouch. In some embodiments, the mouth is defined by an opening within one of, or between two membranes forming the pouch.

In some embodiments, the medication insert area is empty and does not contain any medication therein, and is thereby configured for receiving therein any of a plurality of different medications, allowing for use of the bandage with any of the plurality of different medications for treating any of a plurality of different symptoms. In some such embodiments, the medication insert area is defined by an empty receptacle located on the underside of the flexible fabric. Some such embodiments further include a barrier extending about the medication insert area that prevents medication inserted in the medication insert area from spreading or moving from the treatment area to an unselected area of treatment. In some embodiments, the adhesive surrounds all of the plurality of sides of the medication insert area.

In some embodiments, the multi-use bandage further includes two membranes defining the medication insert area. In some such embodiments, the two membranes define the medication insert area therebetween. In some embodiments, the two membranes include a waterproof membrane and a mesh membrane. In some such embodiments, the waterproof membrane defines a top of the medication insert area and the mesh membrane defines a bottom of the medication insert area. In some embodiments, the two membranes include a non-transparent membrane and a transparent membrane. In some such embodiments, the non-transparent membrane defines a top of the medication insert area and the transparent membrane defines a bottom of the medication insert area. In some embodiments, the non-transparent membrane prevents penetration of a medication received in the medication insert area therethrough, and the transparent membrane allows penetration of a medication received in the medication insert area therethrough and onto the treatment area. In some embodiments, the non-transparent membrane is waterproof and the transparent membrane is a mesh. Preferably, the two membranes are configured to balance the medication received in the pouch and fluids on skin of the treatment area. The two membranes also are preferably configured to maintain a natural moisture and medication balance that allows skin cells to migrate without drying out and to form new skin tissue.

In accordance with another aspect, the present invention is directed to a multi-use bandage comprising: (i) a flexible fabric; (ii) first means located on an underside of the flexible fabric for overlying a treatment area on a user, and for receiving therein any of a plurality of different medications for treating any of a plurality of different symptoms; and (iii) second means located on an underside of the flexible fabric for surrounding a plurality of sides of the first means, for adhesively engaging the user's skin about the treatment area, and for preventing medication inserted in the medication insert area from spreading or moving from the treatment area to an unselected area of treatment.

Some embodiments of the present invention further comprise third means for preventing a medication received in the second means from penetrating through the flexible fabric. Some embodiments further comprise fourth means for slowing the absorption of medication received in the first means into the treatment area. In some embodiments, the first means is a medication insert area located on an underside of the flexible fabric, and the second means is an adhesive. In some embodiments, the first means further includes a pouch defining the medication insert area. In some embodiments, the third means is a waterproof membrane. And in some embodiments, the fourth means is a mesh.

In accordance with another aspect, the present invention is directed to a method comprising the following steps:

(i) inserting a medication into an empty medication insert area located on an underside of a flexible bandage;

(ii) placing the medication insert area and medication received therein onto a treatment area of a user with the medication in contact with the treatment area; and

(iii) adhesively attaching the flexible bandage to a skin of the user about the medication insert area, and preventing the medication from spreading or moving from the treatment area to an unselected area of treatment.

In some embodiments, the method further comprises preventing the medication received in the medication insert area from penetrating into the flexible bandage. In some such embodiments, the preventing includes using a waterproof membrane to prevent the mediation from penetrating into the flexible bandage. In some embodiments, step (i) includes inserting the medication into an empty pouch defining the medication insert area on the underside of the flexible bandage. In some such embodiments, step (i) further includes inserting the medication through a mouth of the empty pouch and into the medication insert area. In some embodiments, step (i) includes inserting the medication into an empty receptacle defining the medication insert area on the underside of the flexible bandage. In some embodiments, step (i) includes inserting the medication between two membranes on the underside of the flexible bandage and defining the medication insert area.

In some embodiments, step (ii) includes placing the medication received in the medication insert area onto a treatment area through a mesh in contact with the treatment area. Some embodiments further comprise balancing the medication and natural fluids on the treatment area and allowing skin cells to migrate without drying out to form new skin tissue. In some embodiments, step (iii) includes adhesively attaching the flexible bandage to the skin along all sides of the medication insert area, and preventing the medication from spreading or moving from the treatment area to an unselected area of treatment. In some embodiments, step (iii) includes preventing the medication from spreading or moving from the treatment area to an unselected area of treatment with a barrier extending about the medication insert area.

One advantage of the present invention is that the medication insert area, such as the pouch, can receive any of a plurality of different medications or other substances, such as any of numerous different creams, ointments, gels, foams or lotions that may be used to treat any of numerous different symptoms. Yet another advantage of the present invention is that the medication received in the medication insert area is prevented from spreading or moving from the treatment area to an unselected area of treatment. In some embodiments of the present invention, the adhesive on the underside of the flexible fabric extends along a plurality of sides or surrounds the medication insert area and prevents such undesirable spreading or moving of the medication away from the treatment area. Yet another advantage of the present invention is that it can minimize the amount of medication needed to treat a symptom because the medication is prevented from spreading or moving away from the treatment area, but rather is maintained on the treatment area. Yet another advantage of the present invention is that the medication may be slowly released from the medication insert area to the treatment area to provide medication over an extended period of time.

Yet another advantage of some embodiments of the present invention is that the waterproof material forming the backside or top of the medication insert area prevents the medication from being absorbed into the flexible bandage and facilitates maintaining the medication on the treatment area. Yet another advantage of some embodiments of the present invention is that the mesh forming the front side or bottom of the medication insert area allows the medication to flow through it and onto the treatment area, while the adhesive about the medication insert area engages the skin and prevents the medication from spreading or moving away from the treatment area. Yet another advantage of such embodiments is that the mesh slows the absorption of medication into the treatment area as compared to embodiments without such a mesh or like membrane.

Yet another advantage of the present invention is that the flexible fabric of the multi-use bandage can come in any of a numerous different shapes and sizes that are currently known, or that later become known, for treating any of a numerous different symptoms. In addition, the multi-use bandages of the present invention can be used to treat any of numerous different conditions or symptoms, in any of numerous different fields, including, for example, podiatry, dermatology, wound care, radiation oncology, and cardiology. For example, in radiation oncology, a Lidocaine gel can be inserted into the medication insert area and applied to a patient's port for treatment of the port with the gel prior to undergoing port access. In cardiology, a nitro paste can be inserted into the medication insert area and applied to the chest or other treatment area of a patient to treat heart attack symptoms. There are numerous exemplary applications in other fields. Yet another advantage of the multi-use bandages of the present invention is that they can be provided in kit form where the kit includes a plurality of such bandages along with a one or more medications for insertion into the medication insert area and application to a treatment area.

Other objects and advantages of the present invention, and/or of the currently preferred embodiments thereof, will become more readily apparent in view of the following detailed description of currently preferred embodiments and accompanying drawings.

### BRIEF DESCRIPTION OF THE DRAWINGS

FIG. 1 is a top plan view of one embodiment of a multi-use bandage including a rectangular adhesive-backed, flexible fabric, and illustrating in broken lines the medication insert area and pouch located on the underside of the flexible fabric;

FIG. 2 is a bottom, perspective view of the multi-use bandage of FIG. 1 showing the pouch on the underside of the flexible fabric forming the medication insert area, the mouth of the pouch, and a medication inserted through the mouth and into the medication insert area;

FIG. 3 is a bottom perspective view of another embodiment of a multi-use bandage where the mouth is further defined by parallel slits in the transparent mesh membrane, and showing a medication inserted from a squeeze tube through the mouth and into the medication insert area of the pouch;

FIG. 4 is a top plan view of another embodiment of a multi-use bandage including a hexagonal adhesive-backed, flexible fabric, and illustrating in broken lines the medication insert area and pouch located on the underside of the flexible fabric;

FIG. 5 is a top plan view of another embodiment of a multi-use bandage including an approximately I-shaped, adhesive-backed, flexible fabric, illustrating partially in broken lines the medication insert area and pouch located on the underside of the flexible fabric, and illustrating the waterproof membrane forming the top or backside of the pouch extending laterally from the sides of the central, reduced width portion of the flexible fabric;

FIG. 6 is a top plan view of the multi-use bandage of FIG. 5 applied to a treatment area on the top or back of a user's hand;

FIG. 7 is a bottom perspective view of another embodiment of a multi-use bandage including a pouch on the underside of the flexible fabric, where the mesh membrane includes a slit in a central region thereof and forming the mouth for inserting therethrough a medication into the medication insert area;

FIG. 8 is a bottom perspective view of another embodiment of a multi-use bandage including a pouch on the underside of the flexible fabric, where the mesh membrane includes a fold or pleat in a central region thereof to facilitate opening the mouth at one end of the pouch and inserting a medication through the mouth and into the medication insert area;

FIG. 9 is a bottom perspective view of another embodiment of a multi-use bandage including a pouch on the underside of the flexible fabric, where the mesh membrane defines two overlapping flaps attached on opposite sides of the medication insert area that can be lifted away from the medication insert area to form the mouth therebetween for inserting a medication through the mouth and into the medication insert area, and showing the releasable backing partially peeled away from the adhesive backing on the underside of the flexible fabric;

FIG. 10 is a bottom perspective view of the multi-use bandage of FIG. 9 showing the flaps of the pouch in an overlapping closing position after receiving a medication in the medication insert area and ready for application to a treatment area;

FIG. 11 is a bottom perspective view of another embodiment of a multi-use bandage where the mesh membrane forms a flap attached along one side of the medication insert area, and an adhesive extends about the border of the medication insert area for attaching the flap thereto after inserting a medication into the medication insert area;

FIG. 12 is a bottom perspective view of the multi-use bandage of FIG. 11 illustrating the insert of medication from a squeeze tube through the mouth defined by the open mesh flap and into the medication insert area; and

FIG. 13 is a cross-sectional view of the multi-use bandage of FIG. 11 taken along line 16-16 of FIG. 11.

### DETAILED DESCRIPTION

The present disclosure describes one or more medical devices, methods and/or techniques for medical treatment. One or more embodiments of the present disclosure describe a medical device that may interface with one or more symptoms (e.g., fungus, soft tissue treatment, skin-related symptoms) to provide treatment with the medical device. One or more embodiments of the present disclosure describe a medical device that allows users (e.g., physicians, patients, etc.) to treat their health symptoms while utilizing medications of their choice. One or more embodiments of the present disclosure describe a medical device that allows users to add medication onto their skin without having the medication rub off from outside factors (e.g., clothing, air dryness, dry skin, etc.). One or more embodiments of the present disclosure describe a medical device that protects an area from germs, dirt, and water that cause infection, while treating the affected area with the medication placed inside the pouch. One or more embodiments of the present disclosure describe a medical device that contains a pouch that allows for, but is not limited to, extra support and protection to eliminate re-injury. One or more embodiments of the present disclosure describe a medical device that allows for treatment of individuals and their respective symptoms by giving them or their physician the freedom to choose what type of medication they would like to use to treat their respective symptom. One or more embodiments of the present disclosure describe a medical device allows for the support of medications include, but are not limited to treatments of wounds, scars, fungus, including nail fungus, acne, arthritis, aches and pains. Uses of this medical device may include, but are not limited to, different types of dermatology treatments. Uses of this medical device may include, but are not limited to, skin dryness, fissures, scars, burns, cuts, rashes, fungus, anti-itch, anti-wrinkle, acne, general skin care, Eczema. The medical bandage may also be used for, but is not limited to, backache, arthritis, muscle strains, cramps, minor aches and pains.

The medical bandage has been developed to provide an environment that maintains a balance of the chosen medication and the fluids on the skin, thereby allowing skin cells to migrate seamlessly without drying out to form new, smooth tissue. The treatment action of the product is due to the balanced environment that the bandage provides allowing the medication to target and treat the affected area in a more direct and efficient manner. In addition, the medical bandage allows the medication to be absorbed to the affected area in a slower fashion, keeping the medication on the affected area three times longer than ordinary medical bandages.

FIGS. 1 and 4 depict illustrations of a top view of embodiments along with the components and features where one or more embodiments of the present disclosure may be useful in such a treatment. It should be understood that the illustration may include additional or fewer components and features than are shown in FIGS. 1 and 4. FIGS. 1 and 4 focus on a portion of what is typically a much larger network of components. The illustrations of FIGS. 1 and 4 reveal three distinct components of the embodiment, the flexible fabric adhesive 1, the medical pouch 5, which cannot be viewed within the illustration due to the angle in which the illustration was drawn, and the area where the mouth 6 of the medical pouch may be located, but is not limited to.

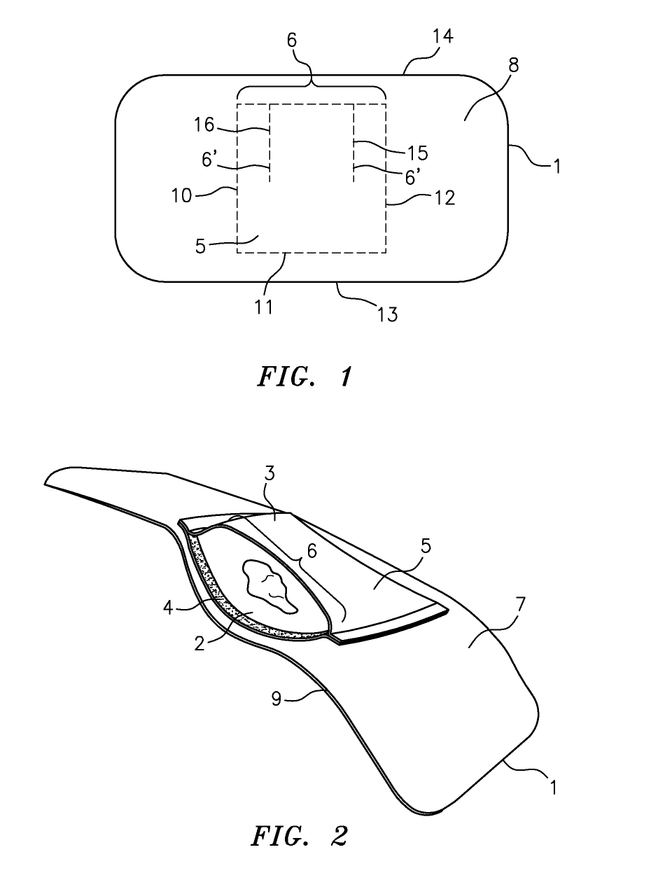

The embodiment is a multi-use, flexible medical bandage. The following depicts the individual components and their respective placement in the embodiment. The embodiment contains a pouch or area to insert medication 5. The pouch/area of insert 5 is made of two membranes. As shown in FIG. 2, the embodiment's pouch/insert area 5 can be made of the following materials, but are not limited to: a transparent membrane 3 (nylon power mesh) and a non-transparent membrane 2 (waterproof polyvinyl chloride, PVC). The top of the pouch/insert area 5 contains a non-transparent membrane 2. The bottom of the pouch/insert area 5 contains a transparent membrane 3. The pouch/insert area 5 is surrounded on the sides and top by a flexible fabric adhesive 1. The adhesive 1 is used for, but is not limited to, the placement of the pouch/insert area 5 on the selected area of the skin. The embodiment may or may not contain an adhesive material on the mouth 6 of the pouch.

FIG. 5 illustrates an example of an embodiment from a top view angle. In relation to FIG. 1, FIG. 5 shows the non-transparent membrane 2, which is one of two membranes that create the embodiment's pouch/insert area.

The embodiment contains a pouch/insert area 5. The pouch/insert area 5 is surrounded on the sides and top by a flexible adhesive 1. This flexible adhesive 1 can but utilized for, but is not limited to, placement of the pouch/insert area 5 on the skin. The embodiment can be operated, but is not limited to operation, in the following order: First, the user of the embodiment opens the pouch/insert area 5 and places the selected medication within the pouch/insert area. Second, after placing the medication within the bandage the user places the bandage so that the mesh material 3 is directly over the selected treatment area. Third, the user may flatten adhesive 1 located around the patch/insert area 5 onto the skin. Once the embodiment is positioned onto the skin the embodiment operates in the following manner: First, an amount of the medication (the amount of medication varies based on the amount inserted by the user) within the pouch/insert area 5 penetrates through the mesh 3 and onto the skin or selected treatment area. The transparent membrane 3 and non-transparent membrane 2 of the pouch/insert area 5 of the embodiment allow the medication to have direct contact with the selected area of treatment for very long periods of time. The transparent membrane 3 and non-transparent membrane 2 of the pouch/insert area 5 of the embodiment allow for no spreading/movement of the medication to unselected areas of treatment. The operation of the embodiment may continue throughout non-physical and physical activity of the user. For example, during physical activity the embodiment provides treatment to the selected area of treatment, as the medication will be able to penetrate through the transparent membrane 3 and onto the selected area of treatment. The operation of the embodiment may or may not include the ability of the user to add medication into the pouch/insert area 5 while the embodiment is still in place on the skin.

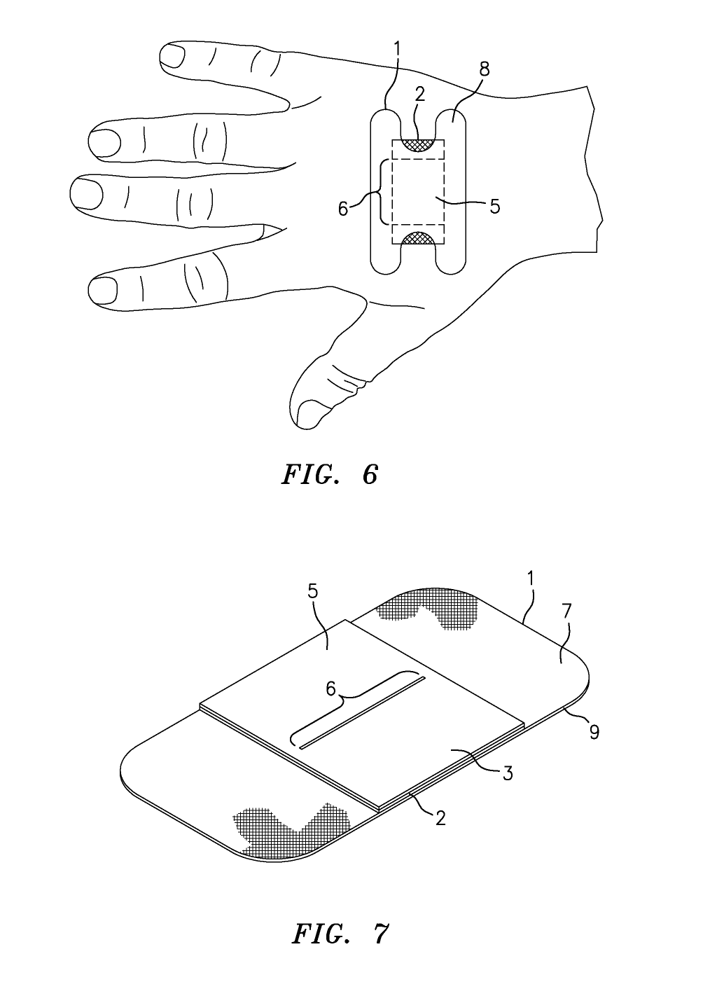

FIG. 6 illustrates an example of the embodiment on a selected area of treatment. FIG. 6 shows where the flexible adhesive 1 of the embodiment can be placed on the skin to allow the embodiment to stay on the selected area during operation. FIG. 6 illustrates an example of the mouth 6 of medical pouch/insert area in relation to the flexible fabric adhesive 1 and a small visible piece of the non-transparent membrane 2 of the medical pouch/insert area.

The pouch of the embodiment maintains a natural moisture and medication balance, allowing skin cells to migrate seamlessly without drying out to form new, smooth tissue. The embodiment can support many different kinds of medications and the respective treatments for multiple symptoms. The embodiment may be used as protection for the selected area of treatment from outside forces (e.g., germs, dirt, and water that cause infection), while treating the selected area of treatment with the selected medication placed inside the pouch/insert area. The embodiment allows for extra support and protection to eliminate re-injury. The embodiment can be used to treat individual patients and their respective symptoms by giving them or their physician the freedom to choose what type of medication they would like to use to treat the symptom. The embodiment may or may not be used under clothing or within footwear/hand wear. The amount of each medication that will be applied with in the pouch/selected area is based upon the amount that is advised by the providers of the medication.

FIG. 2 illustrates the underside of the embodiment of FIG. 1. Component 5 is the medical pouch/insert area. FIG. 2 shows an example of medication within component 5. Component 6 is the mouth of the medical pouch/selected area. Component 4 is the adhesive material that may be placed within component 6. Component 3 is the transparent membrane that makes up part of component 5. The transparent membrane is the component of the embodiment that is placed on top or over the selected area of treatment. Component 2 is the non-transparent membrane that makes up part of component 5. FIG. 2 illustrates component 2 and component 3 as the two main components of the embodiment's medical pouch/insert area.

The embodiments may be constructed in multiple ways, utilizing multiple variations of components. The embodiments can be constructed using different types of materials that make up the two types of membranes of the pouch/insert area 5 within the embodiments. There are multiple combinations of the type of membranes that can be used with different types of shapes and sizes (e.g., combination of transparent and non-transparent materials, square, circle, hexagon, etc.). The embodiments can be constructed utilizing different types of adhesives with different shapes and sizes (e.g., polyester filled transfer adhesive, transparent adhesive with shape of a circle, rectangle, etc.). As stated earlier, the embodiments can be constructed to with many different types of medications (e.g., dermatology, muscle, soft tissue medications, etc.). The embodiments can be constructed in different sizes to be used to cover selected areas of treatment of different sizes.

As stated earlier, the embodiments may be used for different types of dermatology treatments. Uses may include but are not limited to skin dryness, scars, burns, cuts, rashes, fungus, anti-itch, anti-wrinkle, acne, general skin care, and Eczema. The embodiments may be used for, but are not limited to, backache, arthritis, muscle strains, cramps, minor aches and pains. The embodiments may be placed on the lower and upper extremities as well as the stomach and back of the body. The embodiments may act as a protective barrier, that protects the affected area from germs, dirt, and water that may cause infection, while treating the selected area of treatment with the selected medication placed inside the pouch at the same time. The pouch/insert area of the embodiments may allow for extra support and protection to eliminate re-injury. The embodiments may be used to treat individual patients and their respective symptoms by giving them or their physician the freedom to choose what type of medication they would like to use to treat the symptom.

Referring further to FIGS. 1-6, the multi-use medical bandage includes a flexible fabric adhesive 1 suited to contour with the shape of a patient's body or an anatomical surface thereof. The flexible fabric adhesive 1 has a first side 7 for facing the skin of the user and a second side 8 that faces outwardly away from the skin of the user. The first side 7 of the flexible fabric adhesive 1 is coated with an adhesive 9. The flexible fabric adhesive 1 may be any appropriate material used for making medical bandages. An example of a commercially available material that is particularly suitable for use in forming the bandage is 3 M 9926 T tricot fabric tape made by Minnesota Mining & Manufacturing.

A pouch/medication insert area 5 is defined by an empty pouch or other receptacle for receiving a medication. The pouch/insert area 5 is fixedly attached to a portion of the first side 7 of the flexible fabric adhesive 1. In the embodiments shown in FIGS. 1-6, the empty receptacle or pouch/insert area 5 is defined by a first membrane 2 and a second membrane 3. The first membrane 2 is positioned on the surface of the first side 7 of the flexible fabric adhesive 1. The second membrane 3 is positioned over the first membrane 2 and is placed in contact with the skin during use of the bandage. The first and second membranes may be joined and sealed on three sides 10, 11, 12 to define the pouch/insert area 5. The three sides 10, 11, 12 may be joined and sealed by any means known to those skilled in the art, such as with an adhesive or a heat seal. The fourth, unsealed edge of the first and second membranes defines an opening or mouth 6 for inserting the medication or other substance into the pouch/insert area 5.

The first and second membranes of the pouch/insert area 5 may be comprised of any appropriate material. In one embodiment, the first membrane 2 is a non-transparent, waterproof material, such as a plastic membrane. For example, a waterproof polyvinyl chloride membrane may be used. Using a waterproof material for the first membrane can prevent water from entering the pouch/insert area 5 through the flexible fabric adhesive 1 while the bandage is being worn by the user, and can further prevent the medication from seeping the pouch/insert area 5 to the surface of the second side 8 of the bandage.

In one embodiment, the second membrane 3 is a material that is transparent, i.e. at least partially porous, such as a mesh. The mesh may be a nylon mesh or similar material, such as a non-woven fabric. The mesh of the second membrane 3 allows the medication in the pouch/insert area 5 to seep or flow out of the pouch to the surface of the skin in the area being treated. A releasable adhesive may be provided at the mouth 6 of the pouch/insert area 5 to seal the first and second membranes together and hold the mouth 6 of the pouch 5 closed after the medication has been inserted into the pouch.

As illustrated in FIGS. 1-6, in some embodiments, the edges of the mouth 6 and opposing edge 11 of the pouch/insert area 5 do not extend to the outer edges 13, 14 of the flexible fabric adhesive 1. When the bandage is applied to a user, the flexible fabric adhesive surrounding the pouch/insert area 5 adheres to the skin of the user to form a barrier around the medication insert area to prevent the medication from spreading or moving from the treatment area to an unselected area that does not require treatment.

In other embodiments, the pouch/insert area 5 may extend to the edges of the flexible fabric adhesive 2. In these embodiments, the mouth 6 of the pouch/insert area 5 may be sealed closed with a releasable adhesive to seal the first membrane 2 and second membrane 3 together and hold the mouth 6 of the pouch/insert area 5 closed after the medication has been inserted into the pouch. A barrier is then formed by the three sealed sides and the sealed mouth of the medication insert area to prevent the medication from spreading or moving from the treatment area to an unselected area that does not require treatment.

As shown in FIG. 2, the pouch/insert area 5 may be fixedly attached to the first side 7 of the flexible fabric adhesive 1 which contacts the skin of the patient when the bandage is applied. The pouch/insert area 5 may be fixedly attached to the first side 7 of the flexible fabric adhesive 1 by the adhesive coating 9 on the flexible fabric adhesive, or alternatively an additional adhesive or other attachment means such as heat bonding may be used to fixedly attach the pouch/insert area 5 to the first side 7 of the flexible fabric adhesive 1.

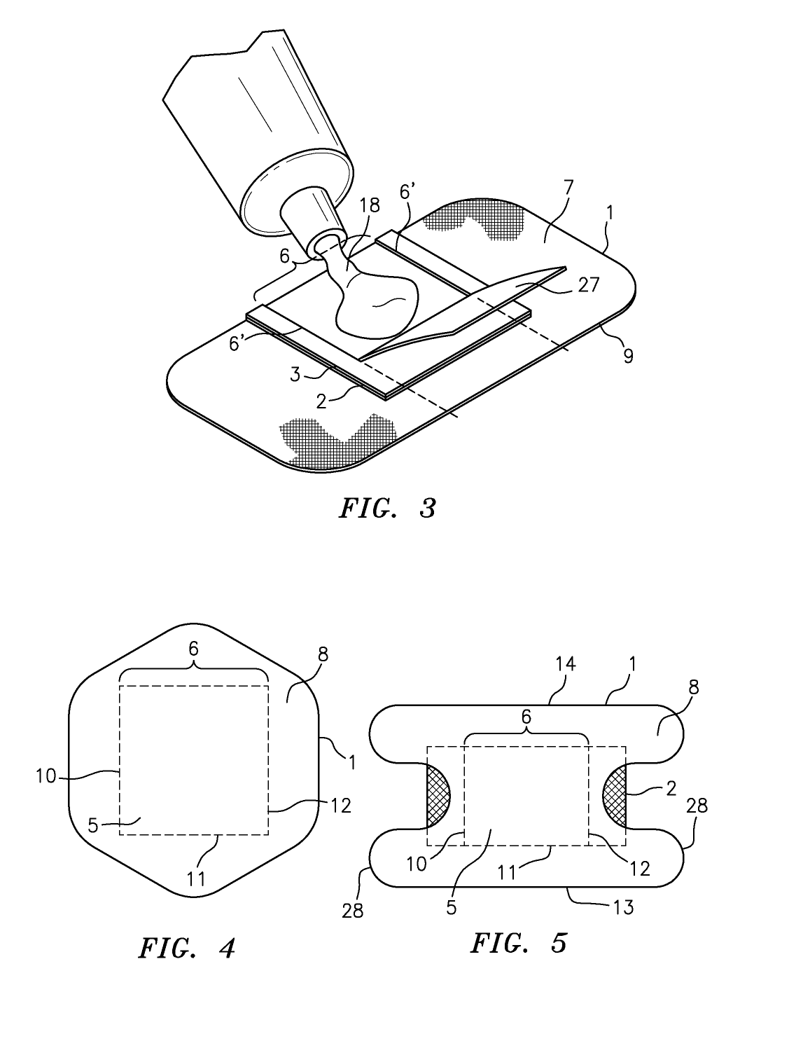

The mouth of the pouch 5 may be formed in several ways. As shown in FIG. 2, the mouth 6 of the pouch 5 may be formed by an opening at an unsealed end of the pouch. Alternatively, the mouth 6 may be further defined by two parallel slits 6′ formed in the second membrane 3. As shown in FIG. 3, the mouth 6 and slits 6′ thereof cooperate to form a flap 27 on the transparent membrane 3. The mouth 6′ may be opened when inserting medication 18, such as an ointment or cream, into the pouch 5. The medication may be provided in an ointment, cream, gel, foam or lotion.

The bandage may be provided with a releasable backing covering the adhesive 9 on the first side 7 of the flexible fabric adhesive 1. The releasable backing may be formed from any of a variety of materials. For example, suitable materials include paper based products (e.g., Kraft paper), polypropylene, polyethylene, polyester or any combination of these materials. The releasable backing can be provided with a release coating (e.g., a silicone release material). The releasable backing may be provided with a scoring line to enable a user to more easily remove the backing from the tape.

The medical bandage may be provided in any desired size or shape for use on various parts of the body, such as a rectangle, square, circle or other shape. For example, as shown in FIGS. 1 and 4, the bandage may be provided in a rectangular shape or hexagonal shape. As shown in FIG. 5, in one embodiment, the medical bandage may be substantially rectangular with areas of material removed to form an approximate I-shape with wing-like ends or elongated tabs 28 for attaching the bandage to the patient.

In use, a medication is inserted into the mouth 6 of the pouch 5. As shown in FIG. 3, in one embodiment, the medication 18 is inserted into the pouch 5 through mouth 6 further defined by slits 6′. After the desired amount of the medication is inserted into the pouch, the mouth 6 is closed, and the bandage is placed on the user with the pouch located over the treatment area. The pouch 5 containing the medication is placed over the treatment area on the skin of the user with the mesh of the second membrane 3 in contact with the skin on the treatment area, and the flexible bandage is adherently attached to the user's skin. The medication seeps or flows though the mesh and onto the skin in the treatment area. The adhesive on the flexible bandage adheres to the user's skin about the pouch in the treatment area to retain the pouch and the medication over the treatment area, while preventing the medication from spreading or moving from the treatment area to an unselected area that does not require treatment.

In another embodiment of the invention shown in FIG. 7, the bandage comprises a flexible fabric adhesive 1, a first side 7 for facing the skin of the user and having an adhesive coating 9, and a second side that faces outwardly away from the skin of the user. The empty receptacle or pouch 5 is defined by a first membrane 2 and a second membrane 3. The first membrane 2 is positioned on the surface of the first side 7 of the flexible fabric adhesive 1. The second membrane 3 is positioned over the first membrane 2 and is placed in contact with the skin during use of the bandage. The first and second membranes are joined and sealed on all four edges to form a barrier to prevent the medication from spreading or moving from the treatment area to an unselected area that does not require treatment. The four edges may be sealed by any means known to those skilled in the art, such as with an adhesive or a heat seal.

The mouth 6 is formed as a slit-like opening in the second membrane 3. In this embodiment, the mouth 6 may be located in the approximate center of the second membrane 3 as shown in FIG. 7, or alternatively the mouth may be formed adjacent and/or otherwise parallel to one of the edges.

In use, the user can spread the mouth 6 of the pouch 5 open to insert or place a quantity of the medication in the form of an ointment, cream, gel, foam or lotion through the mouth and into the medication insert area of the pouch. The mouth 6 is then closed and the bandage is placed on the user with the mesh of the second membrane 3 of the pouch 5 located over the treatment area and the flexible bandage is adherently attached to the user's skin. The medication seeps or flows through the mesh of the second membrane onto the treatment area. The adhesive on the flexible bandage adheres to the user's skin about the pouch in the treatment area to retain the pouch and the medication over the treatment area, while preventing the medication from spreading or moving from the treatment area to an unselected area that does not require treatment.

In another embodiment of the bandage illustrated in FIG. 8, the empty receptacle or pouch defining the medication insert area 5 is formed on the first side 7 of the flexible fabric adhesive 1 by folding the second, transparent membrane 3 to form a pleat 21 on one side of the second membrane 3. The pleat 21 causes the second membrane material opposite the pleat to be elevated and form the empty receptacle that defines the pouch/medication insert area 5. Medication can be inserted into the mouth 6.

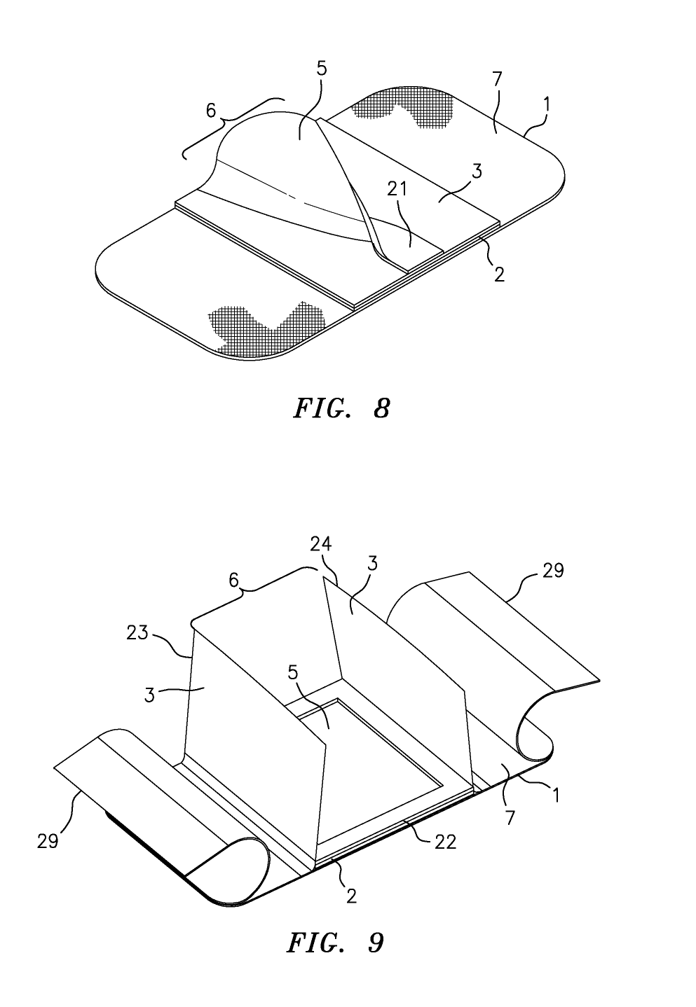

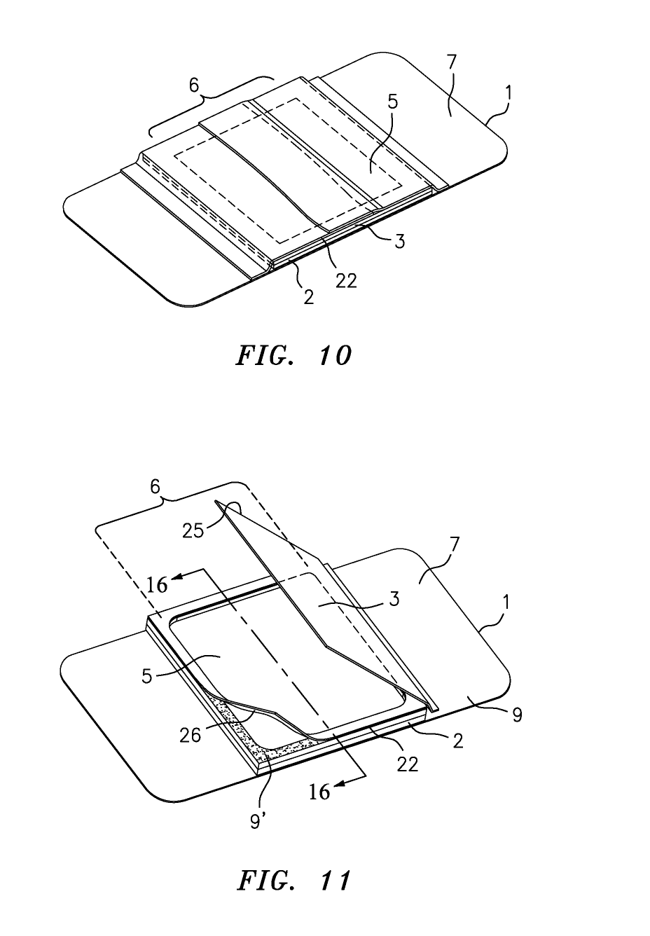

In another embodiment of the bandage illustrated in FIGS. 9-10, the bandage comprises a flexible fabric adhesive 1, a first side 7 for facing the skin of the user and having an adhesive coating, and a second side that faces outwardly away from the skin of the user. A first, non-transparent membrane 2 is positioned on the surface of the first side 7 of the flexible fabric adhesive 1. Similar to the embodiments described above where the pouch/insert area is defined by border of the two membranes, the empty receptacle or pouch 5 is defined by a border 22 attached to the first membrane 2. The border 22 has a sufficient thickness to provide an empty receptacle or pouch 5 for receiving the medication. The border 22 also acts as a barrier to prevent medication inserted into the medication insert area within the border 22 from spreading or moving outside the treatment area. The border 22 may be made using any appropriate material known in the art. In one embodiment, the border using a medical foam having adhesive on two sides. The border may also be any desired shape, such as a square, rectangle or circle.

The second membrane 3 of the flexible bandage is provided in the form of one or more flaps fixedly attached to the first side of the flexible fabric adhesive 1. As shown in FIGS. 9 and 10, in one embodiment, the second membrane 3 comprises two flaps 23, 24 fixedly attached to the flexible fabric adhesive 1 on opposing sides of the border 22. The two flaps 23, 24 may have sufficient length to allow the flaps to overlap in the center of the receptacle formed by the border 22, as shown in FIG. 10. The flaps are comprised of the mesh material of the second membrane 3, such as a nylon mesh or a non-woven fabric. As can be seen in FIG. 10, the border 22 and flaps 23, 24 cooperate to form a pouch defining a medication insert area. Releasable backings 29 are provided to cover the flexible fabric adhesive 1 prior to use.

In use, the desired medication in the form of an ointment, cream, gel, foam or lotion is placed into the medication insert area within the border 22 and the two flaps 23, 24 are closed to cover the medication within the pouch 5. The flaps 23, 24 may be held in place by contact with the medication, such as an ointment, cream, gel, foam or lotion. Alternatively, an adhesive may be provided on the surface of the border to hold the flaps in place after the ointment is placed within the border. Where the border is provided with an adhesive on the surface of the border, the bandage may be provided with a releasable backing covering the adhesive on the border. After the medication is inserted into the medication insert area within the border, the releasable backing may be removed from the border and the flaps closed and adhered to the border.

As shown in FIGS. 11-13, in another embodiment the empty receptacle or pouch 5 defining the medication insert area is formed by a border 22 fixed to the first, non-transparent membrane 2 on the flexible fabric adhesive 1 and defining within it the medication insert area. The second, transparent membrane 3 is provided as a single flap 25 that is attached to the flexible fabric adhesive 1 on one side of the border 22 and extends across the entire width of the border. The border 22 has an adhesive 9′ on the surface, which is covered by a releasable backing 26. As can be seen, the border 22 and flap 25 cooperate to define the pouch 5 with the medication insert area therein.

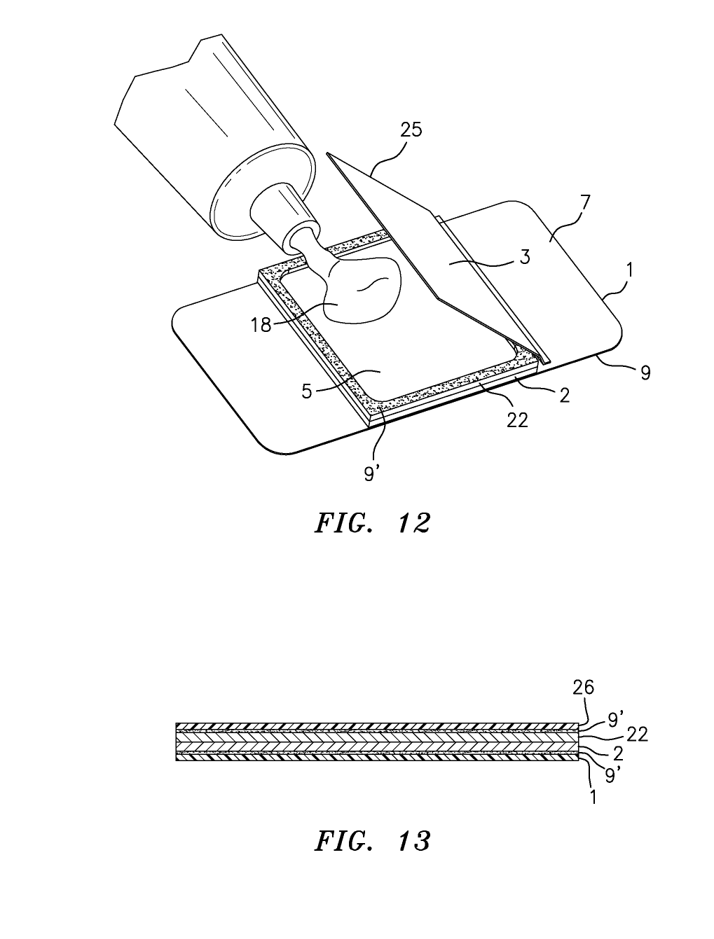

As shown in FIG. 12, in use, medication 18 in the form of an ointment, cream, gel, foam or lotion is inserted into the medication insert area defined by the empty receptacle or pouch within the border. After the medication has been inserted into the medication insert area, the releasable backing 26 is removed and the flap 25 is placed over the border to cover the medication insert area and is adhered to the border by the adhesive 9′ on the surface of the border.

As may be recognized by those of ordinary skill in the pertinent art, numerous changes may be made to the above-described and other embodiments of the present invention without departing from the scope of the invention as defined, for example, in the appended claims. The term pouch is used herein to mean any empty receptacle defining a medication insert area that is configured to receive a medication or other substance, such as a cream, ointment, gel, foam or lotion, on the underside of a flexible bandage, and to prevent the medication or other substance inserted in the medication insert area from spreading or moving from the treatment area to an unselected area of treatment. Accordingly, the pouch may take any of numerous different shapes or configurations, for receiving into the mediation insert area any of numerous different medications or other substances, for treating any of numerous different symptoms or conditions, or for any of numerous different applications, that are currently known or that later become known. A pad or other attachment may be placed on the pouch to prevent direct contact of the mesh with a treatment area, particularly where the treatment area may become irritated by contact with the mesh, for example in treatment of a wart. The flexible fabric, the adhesive, the medication insert area, and the pouch forming the medication insert area, may take the form of any of numerous different materials, sizes, shapes and/or configurations, that are currently known, or that later become known. The flexible fabric adhesive may be any desired color, including any desired shade of tan, or it may be clear to make it less visible when used on exposed areas of skin. Alternatively, the flexible fabric adhesive may have any desired printed pattern on the outer surface. This detailed description of embodiments is therefore to be taken in an illustrative, as opposed to a limiting, sense.

What is claimed is:

1. A multi-use bandage comprising:
a flexible fabric;
a medication insert area located on an underside of the flexible fabric, defining a plurality of sides, and configured for receiving therein any of a plurality of different medications for treating any of a plurality of different symptoms and overlying a treatment area on a user; and
an adhesive located on an underside of the flexible fabric and surrounding a plurality of sides of the medication insert area, wherein the adhesive is configured for adhesively engaging the user's skin about the treatment area, and for preventing medication inserted in the medication insert area from spreading or moving from the treatment area to an unselected area of treatment.

2. A multi-use bandage as defined in claim 1, further including a pouch defining the medication insert area.

3. A multi-use bandage as defined in claim 2, wherein the pouch includes a waterproof surface located on the underside of the flexible fabric, and a mesh overlying the waterproof surface and configured to receive any of the plurality of different medications between the mesh and waterproof surface.

4. A multi-use bandage as defined in claim 3, further comprising a waterproof membrane defining the waterproof surface.

5. A multi-use bandage as defined in claim 3, wherein the mesh forms a flap.

6. A multi-use bandage as defined in claim 3, wherein the pouch includes a mouth.

7. A multi-use bandage as defined in claim 1, wherein the medication insert area is empty and does not contain any medication therein, and is thereby configured for receiving therein any of a plurality of different medications allowing for use of the bandage with any of the plurality of different medications for treating any of a plurality of different symptoms.

8. A multi-use bandage as defined in claim 1, further including two membranes defining the medication insert area.

9. A multi-use bandage as defined in claim 8, wherein the two membranes define the medication insert area therebetween.

10. A multi-use bandage as defined in claim 8, wherein the two membranes include a waterproof membrane and a mesh membrane.

11. A multi-use bandage as defined in claim 10, wherein the waterproof membrane defines a top of the medication insert area and the mesh membrane defines a bottom of the medication insert area.

12. A multi-use bandage as defined in claim 8, wherein the two membranes include a non-transparent membrane and a transparent membrane.

13. A multi-use bandage as defined in claim 12, wherein the non-transparent membrane defines a top of the medication insert area and the transparent membrane defines a bottom of the medication insert area.

14. A multi-use bandage as defined in claim 13, wherein the non-transparent membrane prevents penetration of a medication received in the medication insert area therethrough, and the transparent membrane allows penetration of a medication received in the medication insert area therethrough and onto the treatment area.

15. A multi-use bandage as defined in claim 14, wherein the non-transparent membrane is waterproof and the transparent membrane is a mesh.

16. A multi-use bandage as defined in claim 8, wherein the two membranes are configured to balance the medication received in the pouch and fluids on skin of the treatment area.

17. A multi-use bandage as defined in claim 8, wherein the two membranes are configured to maintain a natural moisture and medication balance that allows skin cells to migrate without drying out and to form new skin tissue.

18. A multi-use bandage as defined in claim 8, wherein the pouch includes a mouth.

19. A multi-use bandage as defined in claim 18, further including an adhesive on the mouth of the pouch.

20. A multi-use bandage as defined in claim 18, wherein the mouth is defined by an opening between the two membranes.

21. A multi-use bandage as defined in claim 1, wherein the adhesive surrounds all sides of the medication insert area.

22. A multi-use bandage as defined in claim 18, wherein the mouth is configured for inserting therethrough a medication into the medication insert area.

23. A multi-use bandage as defined in claim 1, wherein the medication insert area is defined by an empty receptacle located on the underside of the flexible fabric.

24. A multi-use bandage as defined in claim 23, further including a barrier extending about the medication insert area that prevents medication inserted in the medication insert area from spreading or moving from the treatment area to an unselected area of treatment.

25. A multi-use bandage comprising:
a flexible fabric;
first means located on an underside of the flexible fabric for overlying a treatment area on a user and for receiving therein any of a plurality of different medications for treating any of a plurality of different symptoms; and
second means located on an underside of the flexible fabric for surrounding a plurality of sides of the first means, for adhesively engaging the user's skin about the treatment area, and for preventing medication inserted in the medication insert area from spreading or moving from the treatment area to an unselected area of treatment.

26. A multi-use bandage as defined in claim 25, further comprising third means for preventing a medication received in the second means from penetrating through the flexible fabric.

27. A multi-use bandage as defined in claim 25, further comprising fourth means for slowing the absorption of medication received in the first means into the treatment area.

28. A multi-use bandage as defined in claim 25, wherein the first means is a medication insert area located on an underside of the flexible fabric, and the second means is an adhesive.

29. A multi-use bandage as defined in claim 28, wherein the first means further includes a pouch defining the medication insert area.

30. A multi-use bandage as defined in claim 26, wherein the third means is a waterproof membrane.

31. A multi-use bandage as defined in claim 27, wherein the fourth means is a mesh.

32. A method comprising the following steps:
(i) inserting a medication into an empty medication insert area located on an underside of a flexible bandage;
(ii) placing the medication insert area and medication received therein onto a treatment area of a user with the medication in contact with the treatment area; and
(iii) adhesively attaching the flexible bandage to a skin of the user about the medication insert area and preventing the medication from spreading or moving from the treatment area to an unselected area of treatment.

33. A method as defined in claim 32, further comprising preventing the medication received in the medication insert area from penetrating into the flexible bandage.

34. A method as defined in claim 32, wherein the preventing includes using a waterproof membrane to prevent the mediation from penetrating into the flexible bandage.

35. A method as defined in claim 32, wherein step (i) includes inserting the medication into an empty pouch defining the medication insert area on the underside of the flexible bandage.

36. A method as defined in claim 35, wherein step (i) further includes inserting the medication through a mouth of the empty pouch and into the medication insert area.

37. A method as defined in claim 32, wherein step (i) includes inserting the medication into an empty receptacle defining the medication insert area on the underside of the flexible bandage.

38. A method as defined in claim 32, wherein step (i) includes inserting the medication between two membranes on the underside of the flexible bandage and defining the medication insert area.

39. A method as defined in claim 32, wherein step (ii) includes placing the medication received in the medication insert area onto a treatment area through a mesh in contact with the treatment area.

40. A method as defined in claim 32, further comprising balancing the medication and natural fluids on the treatment area and allowing skin cells to migrate without drying out to form new skin tissue.

41. A method as defined in claim 32, wherein step (iii) includes adhesively attaching the flexible bandage to the skin along all sides of the medication insert area and preventing the medication from spreading or moving from the treatment area to an unselected area of treatment.

42. A method as defined in claim 32, wherein step (iii) includes preventing the medication from spreading or moving from the treatment area to an unselected area of treatment with a barrier extending about the medication insert area.
